(19) United States
(12) Patent Application Publication
(10) Pub. No.: US 2022/0143357 A1
(43) Pub. Date: May 12, 2022

(54) AN IMPROVED LIQUID RESERVOIR FOR A HUMIDIFIER, A MEDICAL DEVICE CONTAINING, A HUMIDIFIER CONTAINING, AND A METHOD THEREFOR

(71) Applicants: VINCENT MEDICAL (DONG GUAN) MANUFACTURING CO., LTD., Guangdong (CN); VINCENT MEDICAL (DONGGUAN) TECHNOLOGY CO., LTD., Dongguan, Guangdong (CN)

(72) Inventors: Yu LEI, Guangdong (CN); Jiebing XU, Guangdong (CN); Haibin YU, Guangdong (CN); Zhenxiang HU, Guangdong (CN)

(21) Appl. No.: 17/437,509

(22) PCT Filed: Jun. 6, 2019
(86) PCT No.: PCT/CN2019/090390

§ 371 Date: Sep. 9, 2021

(51) Int. Cl. A61M 16/16; G01F 23/292; A61M 16/00; F24H 15/132

(52) U.S. Cl. CPC A61M 16/162; G01F 23/2922; A61M 16/024; A61M 2205/3653; A61M 2205/3313; A61M 2205/3389; A61M 16/0051; F24H 15/132

(57) ABSTRACT

A liquid reservoir for a humidifier containing a base, a top portion opposite the base, a side wall, and a liquid presence indicator. The side wall contains a lower side wall portion adjacent to the base, and an upper side wall portion adjacent to the top portion. The liquid presence indicator interacts with the humidifier to detect the presence of liquid in the liquid reservoir. A humidifier, and/or a medical device may contain the liquid reservoir, and a method may employ the humidifier and/or medical device.

### FIELD OF THE INVENTION

The present invention relates to medical devices such as humidifiers and liquid reservoirs, and methods for using such devices. More specifically, the present invention relates to improved liquid reservoirs for medical devices and humidifiers that are safer, and methods for using such devices.

### BACKGROUND

Medical devices, such as humidifiers are known to provide humidified air to a user, typically patients in hospitals, hospices, and even at home, so as to prevent illness, soothe symptoms, etc. Humidifiers often contain a liquid reservoir, or a water reservoir, which is placed upon a heating plate and is connected to an air pathway such as a breathing circuit. Typically a blower pushes air through the air pathway, the heater plate heats up the liquid in the liquid chamber, and then the air becomes humidified and then proceeds downstream along the breathing circuit.

One of the major technical challenges with humidifiers is that they need to be able to detect when the liquid reservoir is empty, and/or when the liquid reservoir is not installed properly and/or not present at all. This prevents the heater plate from continuing to heat up, potentially leading to damage, a fire, injury, excessive energy use, etc.

Current methods include the use of weight sensors, electrical sensors, voltage sensors, etc. which tend to be a bit inaccurate, especially in environments with varying conditions such as temperature, ventilation, etc.

Accordingly, there exists a need for improved liquid reservoir presence detection methods and apparatuses, and also for improved liquid presence detection methods and apparatuses. It is also desirable to have more accurate liquid presence detection methods and apparatuses.

### SUMMARY OF THE INVENTION

An embodiment of the present invention relates to a liquid reservoir for a humidifier containing a base, a top portion opposite the base, a side wall, and a liquid presence indicator. The side wall contains a lower side wall portion adjacent to the base, and an upper side wall portion adjacent to the top portion. The liquid presence indicator interacts with the humidifier to detect the presence of liquid in the liquid reservoir.

In an embodiment of the present invention, the present invention relates to a humidifier containing a liquid reservoir as described herein. The humidifier further contains a power source, a controller operatively-connected to the power source, and a liquid presence detector operatively-connected to the controller. The liquid presence detector interacts with the liquid presence indicator to detect the presence of liquid within the liquid reservoir.

In an embodiment herein, a method of use of the humidifier and/or the medical device herein, may further include the steps of providing the humidifier and/or medical device herein, emitting light from the light emitter, reflecting light from the substantially flat surface to form reflected light, detecting the reflected light by the light sensor, generating light sensor data, transmitting the light sensor data to the controller, comparing the light sensor data to a threshold value, and determining whether to terminate power to the heater plate.

Without intending to be limited by theory, it is believed that the present invention may provide one or more benefits such as more accurate liquid detection, a reduced chance of overheating, a reduced chance of heat damage, a reduced chance of fires, a reduced chance of a user/patient receiving unhumidified air, etc.

### BRIEF DESCRIPTION OF THE DRAWINGS

FIG. 1 shows an embodiment of a liquid reservoir according to the present invention;

FIG. 2 shows a top perspective view of an embodiment of a medical device of the present invention, with the liquid chamber removed;

FIG. 3 shows an embodiment of the invention with a close-up view of the detector suite, when removed from the housing FIG. 4 shows a side perspective view of an embodiment of a humidifier containing a liquid reservoir;

FIG. 5a shows a schematic diagram of an embodiment of the liquid presence detection system herein, when the liquid level is too low;

FIG. 5b shows a schematic diagram of an embodiment of the liquid presence detection system herein, when the liquid level is above the prism apex;

FIG. 6 shows a perspective view of an embodiment of a humidifier with a liquid reservoir connected to a breathing circuit;

FIG. 7a shows a schematic diagram of an embodiment of the liquid presence detection system herein, when the liquid level is too low; and

FIG. 7b shows a schematic diagram of an embodiment of the liquid presence detection system herein, when the liquid level is sufficient.

The figures herein are for illustrative purposes only and are not necessarily drawn to scale.

### DESCRIPTION OF THE PREFERRED EMBODIMENTS

Unless otherwise specifically provided, all measurements are made in metric units. Furthermore, all percentages, ratios, etc. herein are by weight, unless specifically indicated otherwise.

Unless otherwise specifically provided, all measurements are made in metric units. Furthermore, all percentages, ratios, etc. herein are by weight, unless specifically indicated otherwise.

As used herein the term “operatively-connected” indicates that the item(s) is(are) connected in a manner which allows them to operate. This may involve, for example, wires, a transmitter/receiver pair, a pair of transceivers, etc. This phrase may also indicate that a physical structure connects the various indicated items.

Unless otherwise specifically described all of the devices, items and/or parts herein may be made from industry-standard materials which are available from multiple suppliers worldwide.

An embodiment of the present invention relates to a liquid reservoir for a humidifier containing a base, a top portion opposite the base, a side wall joining the base and the top portion, and a liquid presence indicator, where the liquid presence indicator contains a substantially flat surface. The side wall contains a lower side wall portion adjacent to the base, and an upper side wall portion adjacent to the top portion. When the liquid presence indicator interacts with the humidifier to detect the presence the liquid in the liquid reservoir.

Typically when the medical device; or the humidifier, does not detect the presence of the liquid reservoir, and/or liquid within the liquid reservoir, then the controller will terminate the power to the heater plate to reduce the chance of burning, damage, fire, and/or excessive energy use.

It is known that the refractive index of air is about 1, while the refractive index of water is about 1.3. In an embodiment herein, the liquid presence indicator contains a substantially flat surface; or a flat surface; or a prism; or contains a material which possesses a refractive index of from about 1.1 to about 1.9; or from about 1.2 to about 1.65, or from about 1.3 to about 1.6; or having a refractive index of from about 1.1 to about 1.9; or from about 1.2 to about 1.65, or from about 1.3 to about 1.6, with respect to the wavelength of light from the light emitter; or the infrared spectrum. In an embodiment herein, the liquid reservoir's side wall is formed of polycarbonate having a refractive index of about 1.6. In an embodiment herein, the substantially flat surface; or the flat surface; or the prism is formed of polycarbonate having a refractive index of about 1.6.

In an embodiment herein, the liquid reservoir's side wall and the substantially flat surface; or the flat surface; or the prism may be formed of, or comprise, the same material. Without intending to be limited by theory, it is believed that such a feature allows easier production and/or moulding of the liquid reservoir, and may be cheaper from both a raw material cost and manufacturing point of view.

In an embodiment herein, the liquid presence indicator is located at least 1 mm above; or from about 1 mm to about 2 cm above; or from about 2 mm above to about 1.5 mm above; or from about 0.5 mm above; to about 1.25 cm above the base of the liquid reservoir. Without intending to be limited by theory, it is believed that as the heater plate will still contain sufficient heat to continue to evaporate the liquid for a short period of time after the power flowing thereto is terminated, then by designing the liquid presence indicator to be above the base of the liquid reservoir, it will reduce the chances of the liquid reservoir running dry and/or overheating due to the lack of liquid; or water. As used herein the liquid presence location is indicated by where the corresponding liquid presence detector is located and at which level it detects a liquid within the liquid reservoir.

In an embodiment herein, the liquid presence indicator reflects and refracts light so as to detect whether or not sufficient liquid is present. In an embodiment herein, the liquid presence detector contains a light emitter and a corresponding light sensor both operatively-connected to the controller. The light emitter emits an amount of light which then impinges upon the liquid reservoir, and is refracted and reflected by the substantially flat surface of the liquid presence indicator by varying amounts depending upon whether liquid; or sufficient liquid, is present in the liquid reservoir. An amount of light is then refracted into the inside of the liquid reservoir, and an amount of light is reflected out of the liquid reservoir, and to the light sensor. The controller calculates the ratio between the amount of light emitted by the light emitter and the amount of light received by the light sensor; or requires a pre-determined threshold of light to be detected by the light sensor, to determine whether liquid; or sufficient liquid, is present in the liquid reservoir.

In an embodiment herein, if the amount of light received by the light sensor is less than 30% of the amount of light emitted by the light emitter, then the controller determines that liquid; or a sufficient amount of liquid, is present in the liquid reservoir. In an embodiment herein, if the amount of light received by the light sensor is 30% or more of the amount of light emitted by the light emitter, then the controller determines that no liquid; or an insufficient amount of liquid, is present in the liquid reservoir.

Without intending to be limited by theory, it is believed that the liquid presence detector herein in conjunction with the liquid reservoir herein reduces the chance of the liquid reservoir running dry, overheating, and/or causing a fire or other hazard. The liquid presence detector and liquid reservoir described herein are easily manufactured, and contain no moving parts, which reduces wear and tear on the parts. Without intending to be limited by theory, it is believed that the liquid presence detector herein in conjunction with the liquid reservoir herein advantageously-provide improvements over, for example, a scale, which contains moving parts. It is believed that the present invention provides long-term, low-maintenance accuracy with a minimum of moving parts and manufacturing complexity and investment.

In an embodiment herein, the liquid reservoir further contains a liquid reservoir presence indicator. The liquid reservoir presence indicator interacts with the humidifier to detect the presence of the liquid reservoir. In an embodiment herein, the liquid reservoir presence indicator is formed of a reflective material, such as, for example, foil, a sticker, and a combination thereof; or an adhesive foil, and a combination thereof; or an adhesive gold foil, an adhesive silver foil, an adhesive mylar foil, and a combination thereof. Such a feature is especially useful when the corresponding liquid reservoir presence detector employs light; or infrared light.

In an embodiment herein, the side wall contains the liquid presence indicator, the liquid reservoir presence indicator, or both the liquid presence indicator and the liquid reservoir presence indicator.

In an embodiment herein, a medical device; or a humidifier; or a medical device containing a humidifier, may contain the liquid reservoir described herein. Specifically in an embodiment herein, the humidifier may further contain a power source, a controller operatively-connected to the power source, an optional heater plate, an optional liquid reservoir presence detector, and a liquid presence detector. The liquid presence detector is operatively-connected to the controller for interacting with the liquid presence indicator to detect the presence of liquid; or sufficient liquid, within the liquid reservoir.

If present, then the heater plate is operatively-connected to the controller, and the base of the liquid reservoir is to be placed on the heater plate. The base may then be heated by the heating plate.

If present, then the liquid reservoir presence detector is operatively-connected to the controller for interacting with the liquid reservoir presence indicator so as to detect the presence of the liquid reservoir.

The controller may control the amount of power flowing to the heater plate. If the liquid presence detector does not detect the presence of liquid; or an insufficient amount of liquid, then the controller may terminate the power source to the heater plate, may initiate an alarm, or otherwise attempt to notify the user/operator/patient. If the optional liquid reservoir presence detector does not detect the presence of the liquid reservoir, then the controller may terminate the power flowing to the heater plate, may initiate an alarm, or otherwise attempt to notify the user/operator/patient.

Only when the liquid presence detector detects the presence of liquid in the liquid reservoir and the optional liquid presence detector detects the presence of the liquid reservoir, does the controller allow power to flow to the heater plate.

In an embodiment herein, the optional liquid reservoir detector does not contain a scale and/or does not operate by weight. In an embodiment herein, the liquid presence detector does not contain a scale and/or does not operate by weight. In an embodiment herein, neither the liquid presence reservoir detector nor the liquid presence detector contains a scale and/or operates by weight.

In an embodiment herein, the optional liquid reservoir presence detector employs light; or a light selected from the group of visible light, infrared light, ultraviolet light, and a combination thereof; or infrared light.

In an embodiment herein, the liquid reservoir presence detector contains a light emitter and a light sensor. The light emitter emits light which then impinges upon the liquid reservoir presence indicator. Or, the light impinges on the liquid reservoir presence indicator and reflects from the liquid reservoir presence indicator. The light sensor then detects the light impinging on the liquid reservoir presence detector, or the light reflected from the liquid reservoir presence indicator. When the liquid reservoir presence detector's light sensor detects the light impinging on the liquid reservoir presence detector, or the light reflected from the liquid reservoir presence indicator, then the controller may maintain the power flowing to the heater plate. However, if the liquid reservoir presence detector's light sensor fails to detect the light impinging on the liquid reservoir presence detector, or fails to detect the light reflected from the liquid reservoir presence indicator, then the controller may terminate the power flowing to the heater plate, may initiate an alarm, or otherwise attempt to notify the user/operator/patient. Without intending to be limited by theory, it is believed that such a feature may prevent the controller from providing power to the heating plate when a liquid reservoir is not present, may indicate to the user/operator/patient to turn off the heater plate and/or to properly affix a liquid reservoir, and therefore prevent overheating of the heater plate so as to reduce the chance for accidents, damage, fire, etc.

In an embodiment herein, the liquid reservoir presence detector and the liquid presence detector are included together in a detector suite, which may reduce the chance of the light emitter and the light sensor from being jostled out of alignment, and may further protect the light emitter and light sensor from damage.

In an embodiment herein, the liquid presence detector employs light; or light selected from the group of visible light, infrared light, ultraviolet light, and a combination thereof; or infrared light; or infrared light of a wavelength of about 760 nm to about 1 mm; or from about 895 nm to about 985 nm.

In an embodiment herein, the liquid presence detector contains a light emitter and a light sensor. The light emitter emits an amount of light which then impinges upon the liquid presence indicator. When the light impinges on the liquid presence indicator a portion of it reflects from the substantially flat surface of the liquid presence indicator; or flat surface and a portion of the light refracts from the substantially flat surface of the liquid presence indicator. The light sensor then detects the amount of light reflecting from the substantially flat surface of the liquid presence indicator and feeds this information to the controller. The controller then uses this information to determine whether liquid; or sufficient liquid, is present in the liquid reservoir. The controller may determine this by, for example, checking whether or not the amount of light detected by the light sensor is above a threshold amount. In an embodiment herein, the threshold amount is 30% or more of the light expected to be emitted by the light emitter.

When the condition of liquid; or sufficient liquid, present in the liquid reservoir is determined by the controller, then the controller maintains the power flowing to the heater plate. However, if the controller determines that no liquid' or insufficient liquid is present in the liquid reservoir, then the controller terminates the power flowing to the heater plate. Without intending to be limited by theory, it is believed that such a feature prevents the controller from providing power to the heating plate when the liquid reservoir is empty of liquid, and therefore prevents overheating of the (empty) liquid reservoir so as to reduce the chance for accidents, damage, fire, etc.

The light emitters and light sensors herein are industry-standard and may be procured from a variety of suppliers worldwide.

In an embodiment herein, the humidifier; or the medical device; or the medical device containing the humidifier, further contains an additional component; or an additional component selected from a clock, an alarm, an breathing circuit, a heating circuit for the breathing circuit, a communication device, and a combination thereof. Such items are standard components and well-known by those skilled in the art.

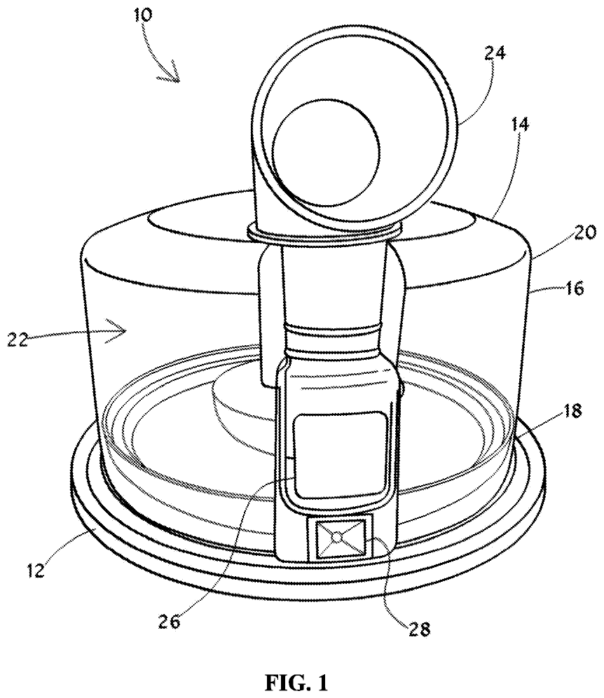

Turning to the figures, FIG. 1 shows an embodiment of a liquid reservoir, 10 according to the present invention. The liquid reservoir, 10, contains a base, 12, and a top portion, 14, opposite the base, 12. A side wall, 16, is perpendicular to the base, 12, and joins the base, 12, with the top portion, 14. A lower side wall portion, 18, is adjacent to the base, 12, while an upper side wall portion, 20, is adjacent to the top portion.

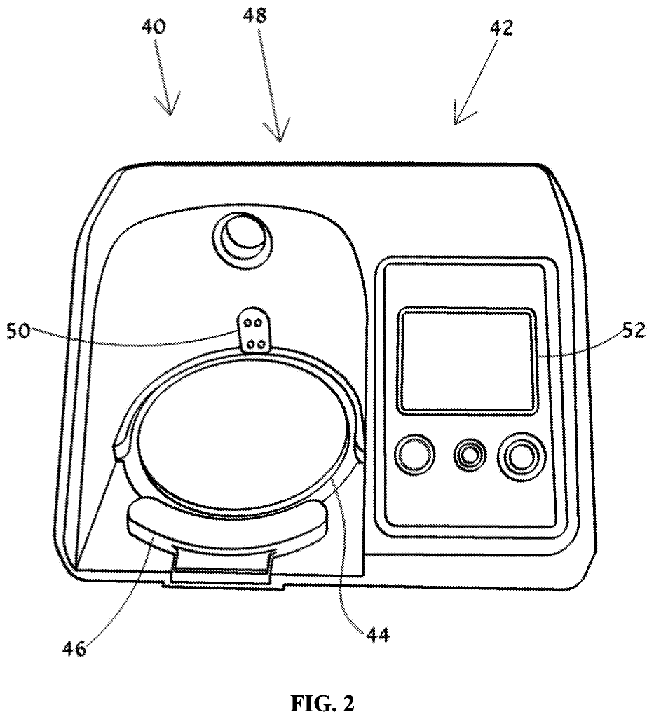

The liquid reservoir herein is typically a transparent or translucent, hollow container, 22, which is made of heat-resistant materials such as high density plastic, glass, metal, and a combination thereof; or polypropylene, polycarbonate, aluminium, and a combination thereof; or polycarbonate, aluminium, and a combination thereof. Typically, the base, 12, contains a metal, such as aluminium, so as to provide both heat resistance as well as good thermal conductivity from the heater plate (see FIG. 2 at 44). the side wall, and the top portion may be formed of a transparent, moulded plastic, such as polycarbonate, and then the metal primped onto the moulded plastic to form a hollow container.

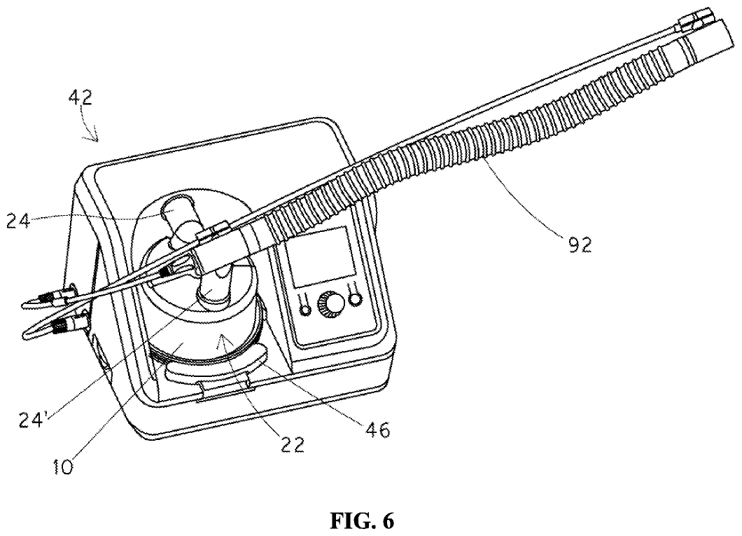

The liquid reservoir contains the liquid; or water; or distilled water; or sterile water, which is evaporated to create the humidified air. The liquid reservoir is operatively-connected; or fluidly-connected, to an air pathway, 24, such as a breathing circuit (see FIG. 6 at 92). He liquid reservoir typically may be opened (see, e.g., FIG. 4 at 24′) so as to allow the user or another person to add a liquid; or water; or distilled water; or sterile water, thereto.

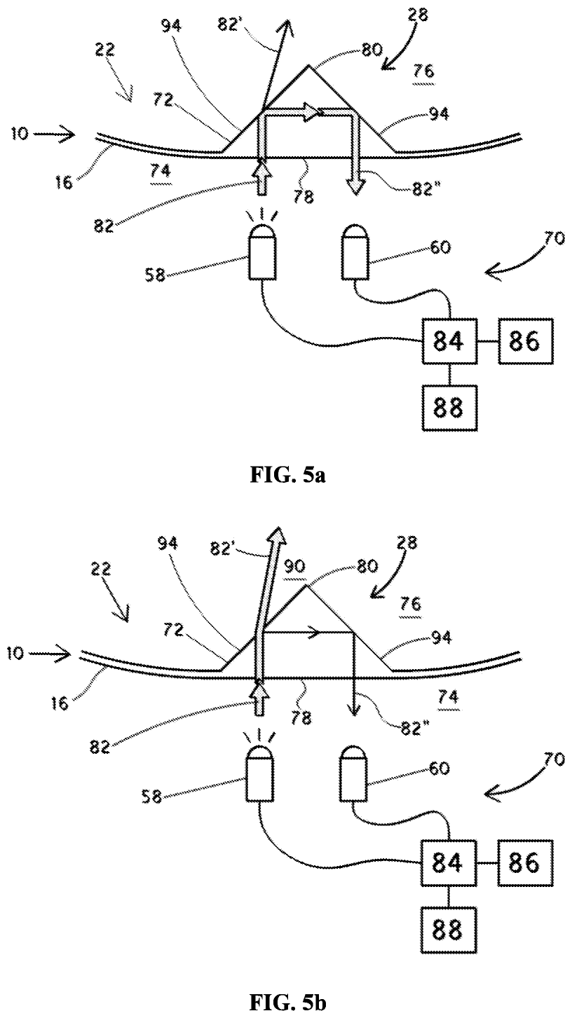

The liquid reservoir, 10, further contains a liquid reservoir presence indicator, 26, on the side wall, 16, as well as a liquid presence indicator, 28, which in this embodiment is a prism (see FIG. 5 at 72) on the side wall, 16. In the embodiment of FIG. 1, the liquid reservoir presence indicator, 26, is a reflective foil sticker, while the liquid presence indicator, 28, is a (pyramidal) prism (see FIG. 5 at 72), with the flat base (see FIG. 5 at 78) embedded into the side wall, 16, and the prism apex (see FIG. 5 at 80) protruding into the hollow container, 22.

As the index of refraction of polycarbonate is similar to that of water, when the liquid reservoir, 10 is filled with water; or distilled water, then as seen in FIG. 5b, a greater proportion of the amount light is refracted into the inside of the liquid reservoir, and a smaller proportion of the amount of light is reflected to the outside of the liquid reservoir, and therefore to the light sensor. However, when the liquid reservoir, 10, is empty of water, as seen in FIG. 5a, or the water level falls below the liquid presence detector (see FIG. 5a at 56)/the prism, 28; or the apex of the prism, 28, then the prism, 28, reflects a greater proportion of the amount of light back to the outside of the liquid reservoir, and therefore to the light sensor, while a smaller proportion of amount of light is refracted into the interior of the liquid reservoir. See FIGS. 5a and 5b and the corresponding description for further explanation.

FIG. 2 shows a top perspective view of an embodiment of a medical device, 40, here a humidifier, 42, of the present invention, with the liquid chamber (see FIG. 1 at 10) removed. The humidifier, 42, shows a heater plate, 44, upon which the base (see FIG. 1 at 12) of the liquid reservoir (see FIG. 1 at 10), would sit. A manual latch, 46, secures the liquid reservoir (see FIG. 1 at 10) to the heater plate, 44. The housing, 48, of the humidifier, 42, further contains a detector suite, 50. A control panel, 52, is also seen in FIG. 2.

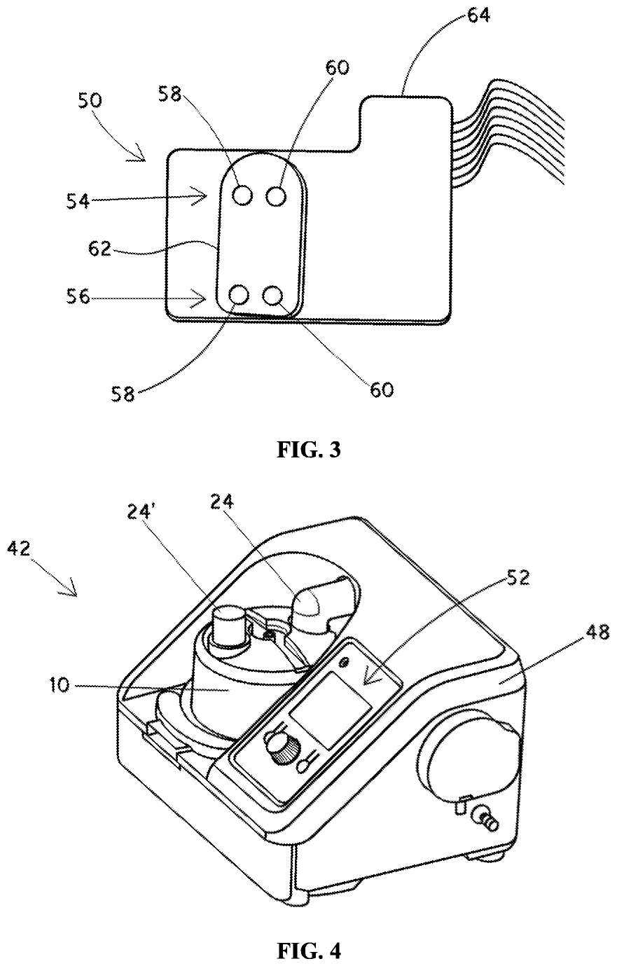

FIG. 3 shows an embodiment of the invention with a close-up view of the detector suite, 50, when removed from the housing (see FIG. 2 at 48). The detector suite, 50, contains a liquid reservoir presence detector, 54, and a liquid presence detector, 56. The liquid reservoir presence detector contains a light emitter, 58, and a light sensor, 60. The liquid presence detector, 56 also contains a light emitter, 58, and a light sensor, 60. A plastic shield, 62, protects the detector suite, and affixes the light emitters, 58, and light sensors, 60, to the circuit board, 64. The plastic shield, 62, also maintains the alignment of the light emitter, 58, and the light sensor, 60, to the proper angles so that they operate correctly with respect to the given liquid reservoir (see FIG. 1 at 10). In an embodiment herein, the light emitter, 58, the light sensor, 60, the plastic shield, 62, and optionally the circuit board, 64, may be securely provided together as a detector suite, 66, which may then reduce the chance of malfunctioning due to, for example, misalignment of the light emitter, 58, and the light sensor, 60.

FIG. 4 shows a side perspective view of an embodiment of a humidifier, 42, containing a liquid reservoir, 10, herein. The liquid reservoir, 10, is securely-installed within the humidifier, 42, and the air pathway, 24, is visible. The air pathway, 24, connects to the housing, 48, which may contain, for example, a blower (not shown), while the other end of the air pathway, 24′, may connect to, for example, a breathing circuit (see FIG. 6 at 92) which is well-known in the art.

FIG. 5a shows a schematic diagram of an embodiment of the liquid presence detection system, 70, herein, when the liquid level is too low. The liquid presence detection system, 70, contains a light emitter, 58, paired with a light sensor, 60. The side wall, 16, contains a prism, 72, embedded therein, which in this view contains at least a pair of substantially flat surfaces, 94. However, as one skilled in the art would understand as the liquid presence indicator, 28, is actually a (pyramidal) prism, 72, the prism actually contains four substantially flat surfaces. The sidewall, 16, separates the outside, 74, from the inside, 76, of the liquid reservoir and therefore inside of the hollow container, 22. The prism, 72, contains a prism base, 78, and a prism apex, 80. The prism base, 78, is embedded in the side wall, 16, of the hollow container (see FIG. 1 at 22), while the prism apex, 80, is inside, 76, of the hollow container (see FIG. 1 at 22).

In FIG. 5a, when the light emitter, 58, emits an amount of light, 82, it enters the prism, 72. As the liquid level is below the prism apex, 80, the index of refraction between the air (about 1) and the prism (about 1.6) is enough to reflect a greater proportion of the amount of light, 82, as indicated by the thicker line of reflected light, 82″, off of the substantially flat surfaces, 94, of the prism, 72, at the prism/air interfaces and return the reflected light, 82″, to the light sensor, 60. A smaller proportion (and therefore thinner line) of refracted light, 82′, refracts from the substantially flat surface, 94, at the prism/air interface into the inside, 76. The amount of the reflected light, 82″, is then detected by the light sensor, 60, and light sensor data is generated and transmitted to the controller, 84. The controller compares the light sensor data to a threshold value; or a pre-determined value; or a percentage of the total amount typically emitted by the light emitter, 58. The controller then determines that the liquid; or liquid level is insufficient/too low, and therefore prevents power from continuing to flow from the power source, 86, to the heater plate, 88.

FIG. 5b shows a schematic diagram of an embodiment of the liquid presence detection system, 70, herein, when the liquid, 90, level is above the prism apex, 80. In this case, the light, 82, emitted from the light emitter, 58, enters the prism, 72, and impinges upon the substantially flat surface, 94, of the prism, 72. As the refractive index of the liquid; or water; or distilled water; or sterilized water, is similar enough to that of the prism, the thicker arrow shows that a greater proportion of the amount of refracted light, 82′, proceeds into the liquid, 84, in the inside, 76 of the liquid reservoir, 10. In contrast, a smaller proportion (and therefore thinner line) of reflected light, 82″, is returned to the light sensor, 60. The amount of the reflected light, 82″, is then detected by the light sensor, 60, and light sensor data is generated and transmitted to the controller, 84. The controller compares the light sensor data to a threshold value; or a pre-determined value; or a percentage of the total amount typically emitted by the light emitter, 58. The controller then determines that the liquid; or liquid level is sufficient, and therefore prevents allows to continue flowing from the power source, 86, to the heater plate, 88.

In the view of FIGS. 5a and 5b, the viewer's perspective is looking downwards from the top portion (see FIG. 1 at 14) towards the base (see FIG. 1 at 12).

FIG. 6 shows a perspective view of an embodiment of a humidifier, 42, with a liquid reservoir, 10, connected to a breathing circuit, 92. The humidifier, 42, is connected to the air pathway, 24, which flows through the liquid reservoir, 10, and the hollow chamber, 22, to the air pathway, 24′, and into the breathing circuit, 92.

The manual latch, 46, keeps the liquid reservoir, 10, securely-attached to the humidifier, 42.

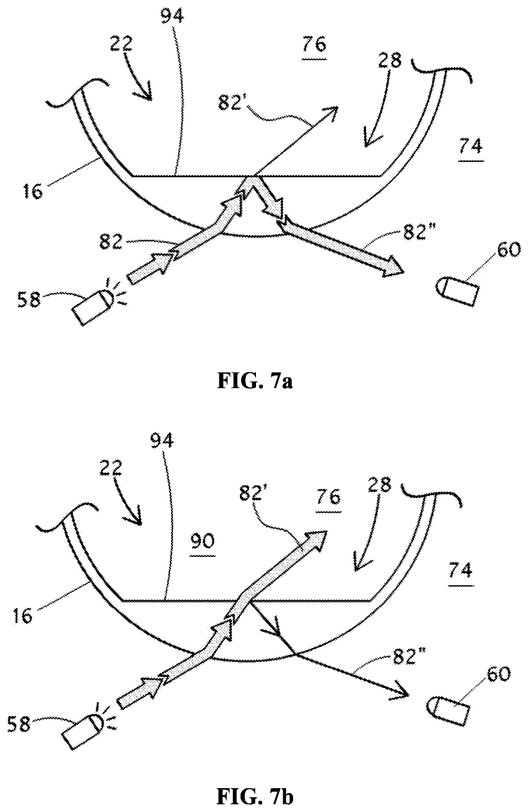

FIG. 7a shows a schematic diagram of an embodiment of the liquid presence detection system, 70, herein, when the liquid level is too low. FIG. 7a is similar to that of FIG. 5a in that a side wall, 16, contains a liquid presence indicator, 28, which contains a substantially flat surface, 94, inside, 76, of the hollow container, 22. An amount of light, 82, emitted by a light emitter, 58, enters the sidewall, 16, is reflected by the substantially flat surface, 94, and then exits the side wall, 16, to the outside, 74, and is detected by the light sensor, 60. While the inventors recognize that the light, 82, refracts (and likely scatters a bit) when entering and exiting the curved sidewall, 16, this is ignored for the purposes of the current discussion. As can be seen in FIG. 7a, as indicated by the thickness of the lines, the amount of reflected light, 82″ is significantly greater in proportion than the amount of refracted light, 82′, and this data is transmitted to the controller (see FIG. 5 at 84). From this, the controller (see FIG. 5a at 84), determines that the liquid level is too low, and therefore prevents power from flowing from the power source (see FIG. 5 at 86), to the heater plate (see FIG. 5 at 88).

In contrast, FIG. 7b shows schematic diagram of an embodiment of the liquid presence detection system herein, when the liquid level is sufficient. FIG. 7b is generally similar to FIG. 5b, in that a side wall, 16, contains a liquid presence indicator, 28, which contains a substantially flat surface, 94, inside, 76, of the hollow container, 22. An amount of light, 82, emitted by a light emitter, 58, enters the sidewall, 16, is reflected by the substantially flat surface, 94, and then exits the side wall, 16, to the outside, 74, and is detected by the light sensor, 60. While the inventors recognize that the light, 82, refracts (and likely scatters a bit) when entering and exiting the curved sidewall, 16, this is ignored for the purposes of the current discussion. As can be seen in FIG. 7b, as indicated by the thickness of the lines, the amount of refracted light, 82′ is significantly greater in proportion than the amount of reflected light, 82″, and this data is transmitted to the controller (see FIG. 5 at 84). From this, the controller (see FIG. 5a at 84), determines that the liquid level is sufficient, and therefore allows power from the power source (see FIG. 5 at 86), to continue flowing to the heater plate (see FIG. 5 at 88).

### Method of Use

In an embodiment of the present invention, the liquid reservoir and the humidifier and/or medical device herein may be used by providing the humidifier described herein, containing the liquid reservoir described herein. The method of use may further include the steps of emitting light from the light emitter, reflecting light from the substantially flat surface to form reflected light, detecting the reflected light by the light sensor, generating light sensor data, transmitting the light sensor data to the controller, comparing the light sensor data to a threshold value, and determining whether to terminate power to the heater plate.

In an embodiment herein, the controller may compare the light sensor data to a threshold value; or a pre-determined value; or a percentage of the total amount typically emitted by the light emitter. The threshold value may be, for example, a pre-determined value; or a percentage of the total amount typically emitted by the light emitter. In an embodiment herein, the threshold value, and/or the pre-determined value may be 30% of the amount of light expected to be emitted by the light emitter. Accordingly, if the amount of light received by the light sensor is less than 30% of the amount of light emitted by the light emitter, then the controller determines that liquid; or a sufficient amount of liquid, is present in the liquid reservoir. In an embodiment herein, if the amount of light received by the light sensor is 30% or more of the amount of light emitted by the light emitter, then the controller determines that no liquid; or an insufficient amount of liquid, is present in the liquid reservoir.

In an embodiment herein, the steps of comparing the light sensor data to a threshold value and determining whether to terminate power to the heater plate are conducted by the controller.

If it is determined, for example, by the controller, that the power to the heater plate should be terminated, then in an embodiment herein the method further contains the step of terminating the power to the heater plate.

In some cases, it may be desirable for the humidifier, or medical device to send a signal, either instead of, or in addition to terminating the power flowing to the heater plate. Accordingly, an embodiment of the present invention further contains the step of transmitting a signal. The signal could be a wireless signal, an electrical signal, etc. as desired, and may be intended to, for example, sound an alarm, notify the user, notify a caregiver, notify a nurse, etc. The signal could be, for example intended to indicate the low liquid level in a hospital system, on a mobile phone app, activate an audible alarm, etc. as desired. In an embodiment herein, the signal may cause the liquid reservoir to automatically fill up again.

Furthermore, it is recognized that the steps of emitting light from the light emitter, reflecting light from the substantially flat surface to form reflected light, detecting the reflected light by the light sensor, generating light sensor data, transmitting the light sensor data to the controller, comparing the light sensor data to a threshold value, and determining whether to terminate power to the heater plate may be repeated; or repeated on a regular basis, to constantly determine whether sufficient liquid is present in the liquid reservoir. Accordingly, in an embodiment of the present invention the steps of emitting light from the light emitter, reflecting light from the substantially flat surface to form reflected light, detecting the reflected light by the light sensor, generating light sensor data, transmitting the light sensor data to the controller, comparing the light sensor data to a threshold value, and determining whether to terminate power to the heater plate are repeated from about 0.1 times per minute (i.e., 1 time per 10 minutes) to about 100 times per minute; or from about 0.2 times per minute (i.e., 1 time per 5 minutes) to about 75 times per minute; or from about 0.5 times per minute (i.e., 1 time per 2 minutes) to about 25 times per minute.

It should be understood that the above only illustrates and describes examples whereby the present invention may be carried out, and that modifications and/or alterations may be made thereto without departing from the spirit of the invention.

It should also be understood that certain features of the invention, which are, for clarity, described in the context of separate embodiments, may also be provided in combination in a single embodiment. Conversely, various features of the invention which are, for brevity, described in the context of a single embodiment, may also be provided separately, or in any suitable subcombination.

All references specifically cited herein are hereby incorporated by reference in their entireties. However, the citation or incorporation of such a reference is not necessarily an admission as to its appropriateness, citability, and/or availability as prior art to/against the present invention.

1. A liquid reservoir for a humidifier comprising:
A) a base;
B) a top portion opposite the base;
C) a side wall joining the base and the top portion, the side wall comprising a lower side wall portion adjacent to the base, and an upper side wall portion adjacent to the top portion; and
D) a liquid presence indicator wherein the liquid presence indicator comprises a substantially flat surface, wherein the liquid presence indicator interacts with the humidifier to detect the presence of liquid in the liquid reservoir.

2. The liquid reservoir according to claim 1, wherein the liquid presence indicator comprises a prism.

3. The liquid reservoir according to claim 2, wherein the liquid presence indicator comprises a material having a refractive index of from about 1.1 to about 1.9; or from about 1.2 to about 1.65, or from about 1.3 to about 1.6; or having a refractive index of from about 1.1 to about 1.9; or from about 1.2 to about 1.65, or from about 1.3 to about 1.6, with respect to the wavelength of light from the light emitter; or the infrared spectrum; and wherein the prism and the side wall comprise the same material.

4. (canceled)

5. The liquid reservoir according to claim 1, further comprising a liquid reservoir presence indicator; wherein the liquid reservoir presence indicator is formed of a reflective material; and wherein the side wall comprises the liquid reservoir presence indicator.

6. (canceled)

7. (canceled)

8. The liquid reservoir according to claim 1, wherein the liquid presence indicator is located at least 1 mm above; or from about 1 mm to about 2 cm above; or from about 2 mm above to about 1.5 mm above; or from about 0.5 mm above; to about 1.25 cm above the base.

9. The liquid reservoir according to claim 1, wherein the side wall comprises the liquid presence indicator.

10. A humidifier comprising the liquid reservoir according to claim 1, wherein the humidifier further comprises:
A) a power source;
B) a controller operatively-connected to the power source; and
C) a liquid presence detector operatively-connected to the controller for interacting with the liquid presence indicator to detect the presence of liquid within the liquid reservoir.

11. The humidifier according to claim 10, wherein the humidifier further comprises a heater plate operatively-connected to the controller, wherein the base is placed on the heater plate and the base may be heated by the heating plate; wherein the controller controls the amount of power flowing to the heater plate; wherein if the liquid presence detector does not detect the presence of liquid, then the controller terminates the power flowing to the heater plate.

12. The humidifier according to claim 10, further comprising a liquid reservoir presence detector operatively-connected to the controller for interacting with the liquid reservoir presence indicator to detect the presence of the liquid reservoir.

13. The humidifier according to claim 10, wherein the humidifier comprises a heater plate operatively-connected to the controller, wherein the base is placed on the heater plate and the base may be heated by the heating plate; wherein the controller controls the amount of power flowing to the heater plate, wherein the humidifier further comprises a liquid reservoir presence detector operatively-connected to the controller for interacting with the liquid reservoir presence indicator to detect the presence of the liquid reservoir; wherein if the liquid reservoir presence detector does not detect the presence of the liquid reservoir, then the controller terminates the power flowing to the heater plate.

14. The humidifier according to claim 12, wherein the liquid reservoir presence detector employs light; or a light selected from the group of visible light, infrared light, ultraviolet light, and a combination thereof; or infrared light; or infrared light of a wavelength of about 760 nm to about 1 mm; or 895 nm to about 985 nm.

15. The humidifier according to claim 14, wherein the liquid reservoir presence detector comprises: A) a light emitter; and B) a light sensor, wherein the light emitter emits light, wherein the light impinges on the liquid reservoir presence indicator; or impinges on the liquid reservoir presence indicator and reflects from the liquid reservoir presence indicator, and wherein when the light sensor detects the light impinging on the liquid reservoir presence indicator; or the light reflected from the liquid reservoir presence indicator, the controller maintains the power flowing to the heater plate, and wherein when the light sensor fails to detect the light impinging on the liquid reservoir presence indicator; or fails to detect reflected from the liquid reservoir presence indicator, the controller terminates the power flowing to the heater plate.

16. The humidifier according to claim 10, wherein the liquid presence detector employs light; or a light selected from the group of visible light, infrared light, ultraviolet light, and a combination thereof; or infrared light; or infrared light of a wavelength of about 760 mm to about 1 mm; or from about 895 nm to about 985 nm.

17. The humidifier according to claim 16, wherein the liquid presence detector comprises: A) a light emitter; and B) a light sensor, wherein the light emitter emits an amount of light, wherein the light impinges on the liquid presence indicator, and wherein when the light sensor detects an insufficient amount of light; or less than 30% of the light emitted by the light emitter, the controller maintains the power flowing to the heater plate, and wherein when the light sensor detects a sufficient amount of light; or 30% or more of the amount of light emitted by the light emitter, the controller terminates the power flowing to the heater plate.

18. The humidifier according to claim 10, further comprising an additional component; or an additional component selected from the group consisting of a clock, an alarm, a breathing circuit, a heating circuit for the breathing circuit, and a combination thereof.

19. A medical device comprising the liquid reservoir according claim 1.

20. A medical device comprising the humidifier according to claim 10.

21. A method of detecting the presence of a liquid; or water, in a liquid reservoir comprising the steps of:
A) providing the humidifier according to claim 10;
B) emitting light from the light emitter;
C) reflecting light from the substantially flat surface to form reflected light;
D) detecting the reflected light by the light sensor;
E) generating light sensor data;
F) transmitting the light sensor data to the controller;
G) comparing the light sensor data to a threshold value; and
H) determining whether to terminate power to the heater plate.

22. The method according to claim 21, wherein the comparing step (G) and the determining step (H) are conducted by the controller.

23. The method according to claim 21, further comprising the step of repeating steps B to H; and wherein the repeating of steps B to H occurs from about 0.1 times per minute to about 100 times per minute; or from about 0.2 times per minute to about 75 times per minute; or from about 0.5 times per minute to about 25 times per minute.

24. (canceled)

25. The method according to claim 21, further comprising the step of: I) terminating power to the heater plate; and further comprising the step of: J) transmitting a signal.

26. (canceled)
